(19) United States
(12) Patent Application Publication
(10) Pub. No.: US 2026/0207198 A1
(43) Pub. Date: Jul. 23, 2026

(54) CLIP DEVICE

(71) Applicant: OLYMPUS MEDICAL SYSTEMS CORP., Tokyo (JP)

(72) Inventors: Shogo Shindo, Hachioji-shi (JP); Naoki Takizawa, Musashino-shi (JP)

(21) Appl. No.: 19/566,666

(22) Filed: Mar. 13, 2026

Related U.S. Application Data
(63) Continuation of application No. PCT/JP2024/032166, filed on Sep. 9, 2024.

(51) Int. Cl. A61B 17/08

(52) U.S. Cl. CPC A61B 17/083

(57) ABSTRACT

A clip device includes a clip unit having arms configured to be opened and closed in a forward direction, a wire connected to the clip unit, and a sheath through which the wire is inserted and extending in a longitudinal axis direction, the clip unit being detachably connected to a distal end portion of the sheath, and the clip unit being able to transition between a first form in which the forward direction of the arm faces in the same direction as the longitudinal axis direction and a second form in which the forward direction of the arms faces in a direction that intersects with the longitudinal axis direction.

### BACKGROUND OF THE INVENTION

### Field of the Invention

The present invention relates to a clip device.

Priority is claimed on US Non-provisional Ser. No. 63/583,011 , filed Sep. 15, 2023, the content of which is incorporated herein by reference.

### DESCRIPTION OF RELATED ART

In endoscopic treatment, medical devices such as clip devices are used to treat biological tissue. These medical devices are introduced into a treatment location using an applicator (introducing device) that can be inserted through a channel of the endoscope.

US Patent Application Publication No. 2021/030425 discloses a clipping device configured to treat biological tissue. In the clipping device disclosed in US Patent Application Publication No. 2021/030425, a control wire configured to control a clip has a bent portion. When the bent portion of the control wire bends, the clip connected to the control wire moves significantly and comes into contact with the tissue.

### SUMMARY OF THE INVENTION

A clip device according to a first aspect of the present invention includes a clip unit having arms configured to be opened and closed in a forward direction, a wire connected to the clip unit, and a sheath, through which the wire is inserted, extending in a longitudinal axis direction, the clip unit is detachably connected to a distal end portion of the sheath, and the clip unit can be transitioned between a first form in which the forward direction of the arms is directed in the same direction as the longitudinal axis direction and a second form in which the forward direction of the arms is directed in a direction intersecting with the longitudinal axis direction.

### BRIEF DESCRIPTION OF THE DRAWINGS

FIG. 1 is a view showing a clip device according to a first embodiment.

FIG. 2 is a perspective view of a clip unit loaded into an applicator in the clip device.

FIG. 3 is a perspective view of the clip unit loaded into the applicator.

FIG. 4 is a cross-sectional view of a distal end portion of the clip device.

FIG. 5 is a view showing a clip locked in a closed form.

FIG. 6 is a view showing transition between forms of the clip unit.

FIG. 7 is a view showing the clip unit configured to treat tissue by tangential approach.

FIG. 8 is a view showing the clip unit with a pair of arms that are swung.

FIG. 9 is a view showing a locking step by the clip unit.

FIG. 10 is a view showing a separating step by the clip unit.

FIG. 11 is a view showing a variant of a coupler in the clip device.

FIG. 12 is a view showing another variant of the coupler.

FIG. 13 is a view showing another variant of the coupler.

FIG. 14 is a view showing another variant of the coupler.

FIG. 15 is a view showing a clip device according to a second embodiment.

FIG. 16 is a cross-sectional view of a distal end portion of the clip device.

FIG. 17 is a view showing transition between forms of the clip unit of the clip device.

FIG. 18 is a view showing the clip unit configured to treat tissue by tangential approach.

FIG. 19 is a view showing the clip unit with a pair of arms that are swung.

FIG. 20 is a view showing a locking step by the clip unit.

FIG. 21 is a view showing a separating step by the clip unit.

FIG. 22 is a view showing a variant of a retaining pipe of the clip unit.

FIG. 23 is a view showing the variant of the retaining pipe.

FIG. 24 is a view showing a procedure using an outer sheath.

FIG. 25 is a view showing a procedure using the outer sheath.

FIG. 26 is a view showing a procedure using the outer sheath.

FIG. 27 is a view showing a procedure using the outer sheath.

FIG. 28 is a view showing a clip device according to a third embodiment.

FIG. 29 is a view showing the clip device.

FIG. 30 is a view showing another aspect of a flexible member of the clip device.

FIG. 31 is a view showing the other aspect.

FIG. 32 is a view showing a variant of a curved member of the clip device.

FIG. 33 is a view showing another variant of the curved member.

FIG. 34 is a view showing another variant of the curved member.

FIG. 35 is a view showing another variant of the curved member.

### DETAILED DESCRIPTION OF THE INVENTION

### First Embodiment

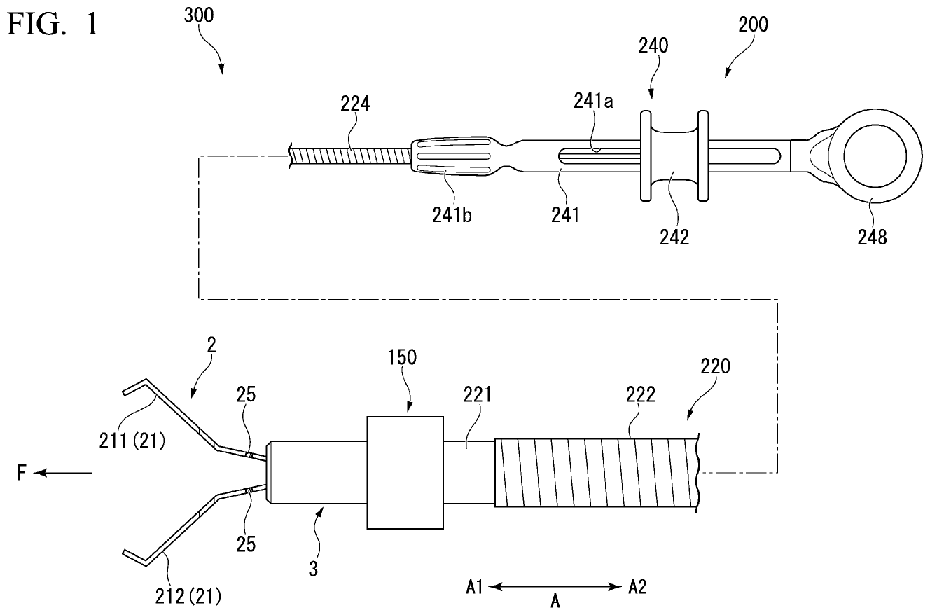

A first embodiment of the present invention will be described with reference to FIG. 1 to FIG. 10.

FIG. 1 is a view showing a clip device 300 according to the embodiment.

The clip device (a clip system, a treatment tool for an endoscope) 300 includes a clip unit 100, a coupler (connecting member) 150, and an applicator 200. The clip unit 100 is loaded into the applicator 200 through which a channel of an endoscope can be inserted, and introduced into a treatment location.

The clip device 300 may be capable of reloading the clip unit 100 into the applicator 200. In this case, a user can remove the used clip unit 100 from the applicator 200 and reload an unused clip unit 100 into the applicator 200.

In the following description, the side of the clip unit 100 of the clip device 300 in a longitudinal direction A is referred to as a distal end side (distal side) A1 of the clip device 300, and the side of a manipulation part 240 of the applicator 200 is referred to as a proximal end side (proximal side) A2 of the clip device 300.

### Applicator 200

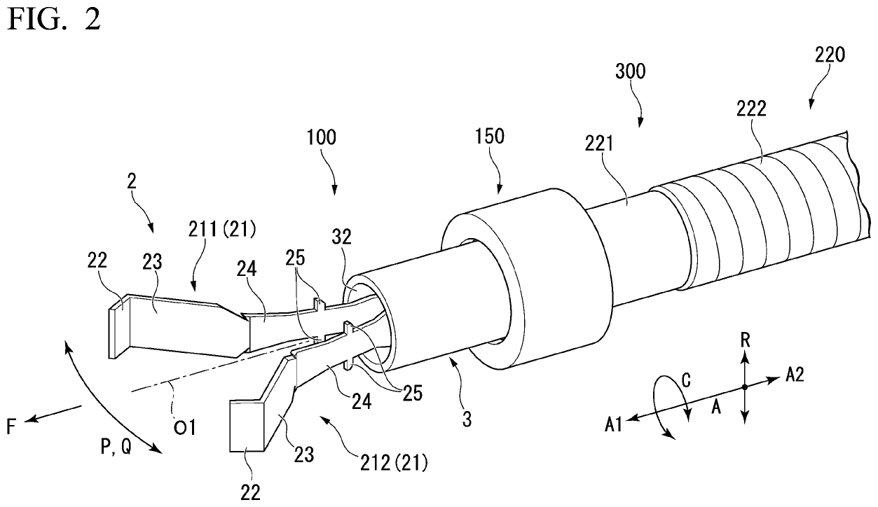

FIG. 2 is a perspective view of the clip unit 100 loaded into the applicator 200.

The applicator (clip unit introducing device) 200 includes a sheath 220, a manipulation wire 230, and the manipulation part 240. The applicator 200 is inserted through, for example, a treatment tool insertion channel of the endoscope, and used in combination with the endoscope. For this reason, the sheath 220 is formed to be sufficiently longer than the length of the treatment tool insertion channel of the endoscope. The sheath 220 is flexible and can be curved to match the curvature of the insertion part of the endoscope.

The sheath 220 includes a distal end tip 221, a distal end-side coil 222, and a hand-side coil 224, and is formed into an elongated tubular shape as a whole. The distal end-side coil 222 is disposed on the side of the distal end portion of the sheath 220. Further, the distal end-side coil 222 and the hand-side coil 224 may be integral.

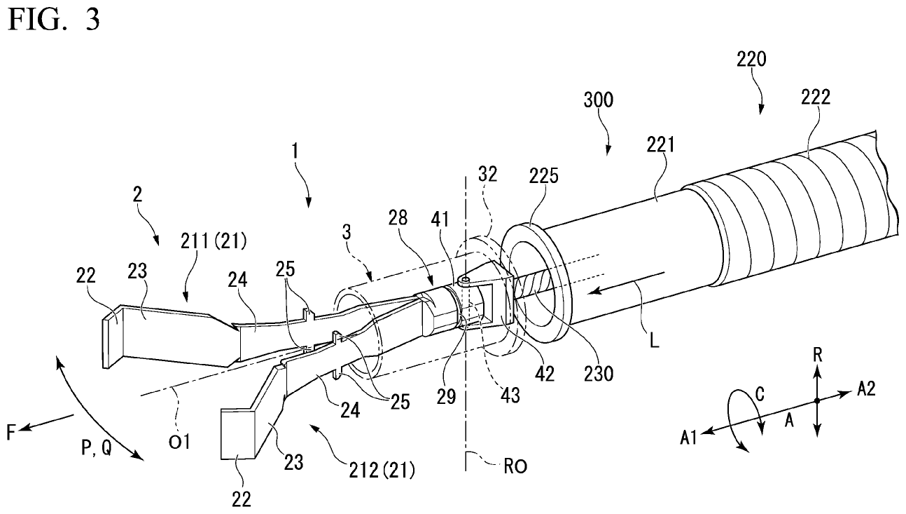

FIG. 3 is a perspective view of the clip unit 100 loaded into the applicator 200. In FIG. 3, the coupler 150 is not shown. The distal end tip (distal end portion) 221 is formed in a cylindrical shape and is disposed at the distal end portion of the distal end-side coil 222. An engaging portion 225 is formed at a distal end of the distal end tip 221. The engaging portion 225 is a convex portion that protrudes outward in a radial direction R perpendicular to the longitudinal direction A. The engaging portion 225 is provided along the entire circumference of the distal end tip 221 in a circumferential direction C.

The manipulation wire (power transmission member) 230 is inserted through the sheath 220 and attached to the clip unit 100 and the manipulation part 240. As shown in FIG. 3, the manipulation wire 230 can protrude from the distal end tip (distal end portion) 221 to the distal end side. The manipulation wire 230 advances and retracts the distal end tip (distal end portion) 221 along a longitudinal axis direction L of the sheath 220 to transmit power to a clip 2 of the clip unit 100.

The manipulation part 240 includes a manipulation main body 241, a slider 242, and a thumb ring 248. The manipulation main body 241, the slider 242 and the thumb ring 248 are injection-molded by, for example, a resin material. The manipulation main body 241 includes a slit portion 241a, and a rotating grip 241b at the distal end side. The slit portion 241a supports the slider 242 to enable advance and retraction.

The slider 242 is attached so as to enable advance and retraction in the longitudinal axis direction of the manipulation main body 241, and the proximal end of the manipulation wire 230 is attached to the slider 242. As the slider 242 advances and retracts along the manipulation main body 241, the manipulation wire 230 advances and retracts relative to the sheath 220.

The thumb ring 248 is attached to the proximal end of the manipulation main body 241 so as to be rotatable around the longitudinal axis of the manipulation main body 241.

### Clip Unit 100

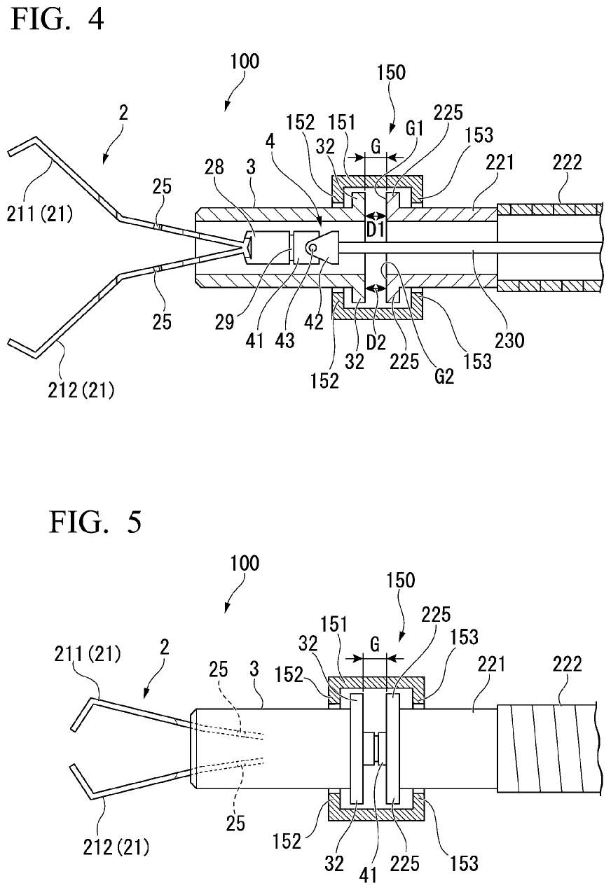

FIG. 4 is a cross-sectional view of the distal end portion of the clip device 300.

The clip unit 100 is loaded onto the distal end tip (distal end portion) 221 of the sheath 220 of the applicator 200 via the coupler (connecting member) 150. The clip unit 100 includes the clip 2, the retaining pipe (tubular member) 3, and a curved member 4.

The clip (clip arm) 2 has a pair of arms 21, and a support portion 28 configured to support the pair of arms 21. The support portion 28 is attached to a distal end of the curved member 4.

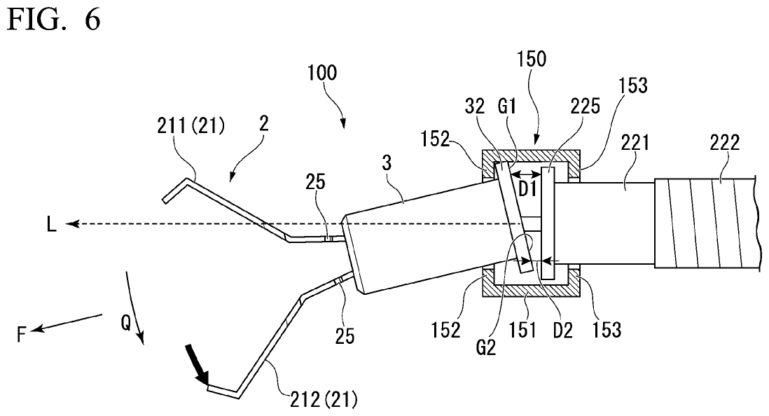

The pair of arms 21 can be opened and closed in a forward direction F, and can transition between an open form (open state) and a closed form (closed state). The pair of arms 21 can swing in a swing direction Q. The pair of arms 21 swings to change the forward direction F (see FIG. 6). As shown in FIG. 2 and FIG. 3, the position of the pair of arms 21 in which the forward direction F approximately coincides with the longitudinal axis direction L of the sheath 220 is referred to as “an initial position” of the pair of arms 21.

The pair of arms 21 have a first arm 211 and a second arm 212. As shown in FIG. 2 and FIG. 3, when the first arm 211 and the second arm 212 are positioned in the “initial position” where they are not swung, they are positioned on either side sandwiching a center axis O1 in the longitudinal direction A. Further, the clip 2 may have three or more arms.

The first arm 211 and the second arm 212 have claws 22, gripping portions 23, sliding portions 24, and engaging portions 25 from a distal end side A1 toward a proximal end side A2.

The claws 22 are formed by bending the distal ends of the first arm 211 and the second arm 212 inward. The gripping portions 23 are formed in a flat plate shape and are portions that grip the tissue. The sliding portions 24 are portions that undergo elastic deformation when the pair of arms 21 are pulled into a retaining pipe 3.

The engaging portions 25 are portions that can engage with an inner circumferential surface of the retaining pipe 3. The engaging portions 25 are convex portions that protrude from the sliding portions 24. Specifically, the engaging portions 25 protrude on either side in an upward/downward direction perpendicular to the longitudinal direction A and an opening/closing direction P of the pair of arms 21.

FIG. 5 is a view showing the clip 2 locked in a closed form.

The engaging portions 25 and the inner circumferential surface of the retaining pipe 3 constitute a locking mechanism configured to lock the clip 2 in the closed form. When the pair of arms 21 are locked in the closed form by the locking mechanism, the pair of arms 21 cannot return to the open form. Further, the locking mechanism is not limited to the combination of the engaging portions 25 of the clip 2 and the inner circumferential surface of the retaining pipe 3, but may have other configurations.

The support portion 28 supports the pair of arms 21 (the first arm 211 and the second arm 212) so that they can be opened and closed freely in the opening/closing direction P. The pair of arms 21, supported by the support portion 28, are biased into the open form. For this reason, the pair of arms 21 of the clip 2 have self-expanding power in the opening/closing direction P.

The support portion 28 has a separating portion 29 that is detachably connected to a distal end member 41 of the curved member 4 at the proximal end side A2. For example, the separating portion 29 may be a breaking portion that breaks when a predetermined breaking force or more is applied due to tension. In addition, the separating portion 29 may be a deforming portion formed in a hook form or the like, which separates from the distal end member 41 by plastic deformation.

The retaining pipe (tubular member) 3 is a tubular member capable of storing at least a portion of the clip 2. The retaining pipe 3 has an internal space 38 through which the clip 2 advances and retracts in the longitudinal direction A. The retaining pipe 3 can fix the clip 2, which is pulled into the internal space 38, in a closed state.

An engaging portion 32 is formed at the proximal end of the retaining pipe 3. The engaging portion 32 is a convex portion that protrudes outward in the radial direction R, perpendicular to the longitudinal direction A. The engaging portion 32 is provided along the circumferential direction C around the entire circumference of the retaining pipe 3.

The curved member 4 supports the pair of arms 21 so that they can swing (rotate) around a rotation axis RO that is approximately perpendicular to the longitudinal direction A. In the embodiment, the swing direction (rotation direction) Q of the pair of arms 21, with the rotation axis RO as the center of rotation, is approximately the same as the opening/closing direction P of the pair of arms 21. That is, a plane in which the pair of arms 21 swing (a plane through which the pair of arms 21 pass as they swing) and a plane in which the pair of arms 21 open and close (a plane through which the pair of arms 21 pass as they open and close) approximately coincide with each other.

The curved member 4 causes the pair of arms 21 to swing when a force greater than or equal to a predetermined value is applied to at least one of the first arm 211 and the second arm 212, such as when at least one of the first arm 211 and the second arm 212 comes into contact with biological tissue or when an external force greater than or equal to a predetermined value is applied to at least one of the first arm 211 and the second arm 212. Otherwise, the support portion 28 does not allow the pair of arms 21 to swing.

The curved member 4 has the distal end member 41, a proximal end member 42, and a hinge (pivoting portion) 43 pivotably connecting the distal end member 41 and the proximal end member 42. The proximal end member 42 is undetachably attached to the manipulation wire 230.

The hinge (pivoting portion) 43 connects the distal end member 41 and the proximal end member 42 so that the distal end member 41 pivots in the swing direction (rotation direction) Q relative to the proximal end member 42.

### Coupler (Connecting Member) 150

The coupler (connecting member) 150 detachably connects the retaining pipe 3 and the sheath 220. The coupler 150 connects the retaining pipe 3 and the sheath 220 so that a gap (rattle) G in the longitudinal axis direction L is formed between the retaining pipe 3 and the sheath 220. The coupler 150 is formed in a cylindrical shape, and has a cylindrical portion 151, a distal end engaging portion 152, and a proximal end engaging portion 153.

The distal end engaging portion 152 is provided at the distal end of the cylindrical portion 151 and is a convex portion that protrudes inward in the radial direction R perpendicular to the longitudinal direction A. The distal end engaging portion 152 is disposed along the entire circumference of the cylindrical portion 151 in the circumferential direction C. The distal end engaging portion 152 engages with the engaging portion 32 of the retaining pipe 3, thereby connecting the coupler 150 and the retaining pipe 3.

The proximal end engaging portion 153 is provided at the proximal end of the cylindrical portion 151 and is a convex portion that protrudes inward in the radial direction R perpendicular to the longitudinal direction A. The proximal end engaging portion 153 is disposed along the entire circumference of the cylindrical portion 151 in the circumferential direction C. The proximal end engaging portion 153 engages with the engaging portion 225 of the sheath 220, thereby connecting the coupler 150 and the sheath 220.

At least one of the distal end side and the proximal end side of the coupler 150 is capable of deformation (elastic deformation, plastic deformation). The deformation of the coupler 150 allows the retaining pipe 3 and the sheath 220 to separate.

FIG. 6 is a view showing transition between forms of the clip unit 100.

The clip unit 100 can transition between the linear form (first form) shown in FIGS. 2 and 3 and the swing form (second form) shown in FIG. 6.

As shown in FIG. 2 and FIG. 3, the linear form (first form) is a form of the clip unit 100 in which the forward direction F of the pair of arms 21 is oriented in the same direction as the longitudinal axis direction L of the sheath 220.

As shown in FIG. 6, the swing form (second form) is a form of the clip unit 100 in which the forward direction F of the pair of arms 21 is oriented in a direction that intersects with the longitudinal axis direction L of the sheath 220. When a force greater than or equal to a predetermined value is applied to at least one of the first arm 211 and the second arm 212, the clip unit 100 swings in the swing direction Q and transitions to the swing form (second form). Specifically, as the pair of arms 21 swings in the swing direction Q, the retaining pipe 3 in contact with the pair of arms 21 moves relative to the sheath 220. When the clip unit 100 is at least in the linear form (first form), the gap (rattle) G in the longitudinal axial direction L is formed between the engaging portion 32 of the retaining pipe 3 and the engaging portion 225 of the sheath 220, which are connected by the coupler 150. For this reason, the retaining pipe 3 can be inclined relative to a direction intersecting with the longitudinal axis direction L of the sheath 220. Further, when the clip unit 100 is in the second form, the engaging portion 32 of the retaining pipe 3 and the engaging portion 225 of the sheath 220 connected by the coupler 150 may be in contact with each other.

As shown in FIG. 4, the gaps G located at positions sandwiching the manipulation wire 230 are referred to as a first gap G1 and a second gap G2. When the clip unit 100 is in the linear form (first form), a first distance D1 of the first gap G1 and a second distance D2 of the second gap G2 are approximately the same. As shown in FIG. 6, when the clip unit 100 is in the swing form (second form), the first distance D1 and the second distance D2 are different. Specifically, when the clip unit 100 transitions from the linear form (first form) to the swing form (second form), one of the first distance D1 and the second distance D2 increases, and the other of the first distance D1 and the second distance D2 decreases.

The clip unit 100 can also transition from the swing form (second form) to the linear form (first form).

### Operation and Action of Clip Device 300

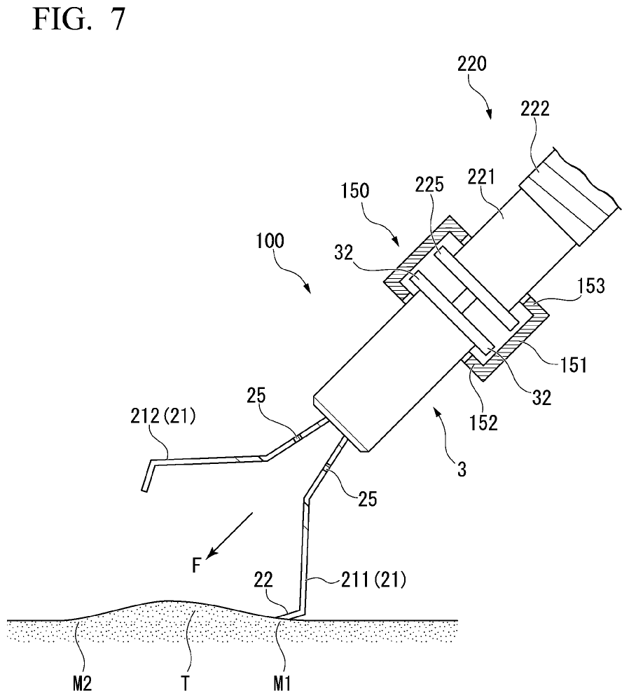

Next, operations and actions of the clip device 300 will be described with reference to FIG. 7 to FIG. 10.

### Approaching Step

A surgeon introduces the clip unit 100 loaded into the applicator 200 into the body via the channel of the endoscope. The surgeon moves the endoscope or the sheath 220 to bring the clip 2 of the clip unit 100 closer to tissue T, which is a treatment region.

There are at least two ways to approach the treatment region with the clip unit 100: a frontal approach and a tangential approach. In the frontal approach, the surgeon manipulates the endoscope or the sheath 220 to dispose the clip unit 100 so that the longitudinal direction A of the clip unit 100 is approximately perpendicular to the surface of the treatment region. In the tangential approach, the surgeon manipulates the endoscope or the sheath 220 to dispose the clip unit 100 so that the longitudinal direction A of the clip unit 100 is along the surface of the treatment region.

### Tangential Approach

FIG. 7 is a view showing the clip unit 100 configured to treat the tissue T by the tangential approach. The surgeon approaches the tissue T, which is the treatment region, with the clip unit 100 in the linear form using the tangential approach. The surgeon hooks the claw 22 of the first arm 211 onto a mucous membrane on the endoscope side (front side) of a mucous membrane M1 and a second mucous membrane M2 (in FIG. 7, the first mucous membrane M1), which are located on either side sandwiching the center of the tissue T.

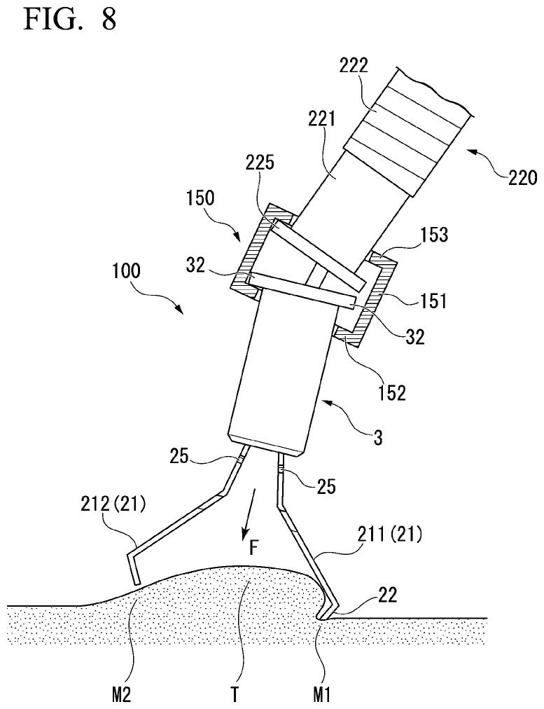

FIG. 8 is a view showing the clip unit 100 with the pair of arms 21 swung. The surgeon further moves the clip unit 100, with the claws 22 hooked onto the first mucous membrane M1, further closer to the tissue T, which is the treatment region. Specifically, the surgeon advances the applicator 200 against the channel of the endoscope. Alternatively, the surgeon further presses the clip unit 100 against the tissue T by advancing the endoscope. As a result, as shown in FIG. 8, the pair of arms 21 swings around the rotation axis RO, and the clip unit 100 takes on the swing form. When the second arm 212 comes into contact with the second mucous membrane M2, the pair of arms 21 are disposed so that the forward direction F faces the front surface of the tissue T, which is the treatment region. The first mucous membrane M1 and the second mucous membrane M2 are grasped by the pair of arms 21.

### Pulling Step

The user retracts the manipulation wire 230 by retracting the slider 242 along the manipulation main body 241. The curved member 4 connected to the manipulation wire 230 pulls the clip 2. As the clip 2 is pulled to the proximal end side A2, the pair of arms 21 are pulled into the retaining pipe 3, and the pair of arms 21 that engage with the distal end opening 3a gradually closes. In this state, when the pulling force of the manipulation wire 230 is released, the self-expanding power of the pair of arms 21 causes the clip 2 to move to the distal end side A1 and return to the open state. The user can grasp the tissue T again (reopening) by returning the pair of arms 21 to the open state.

### Locking Step

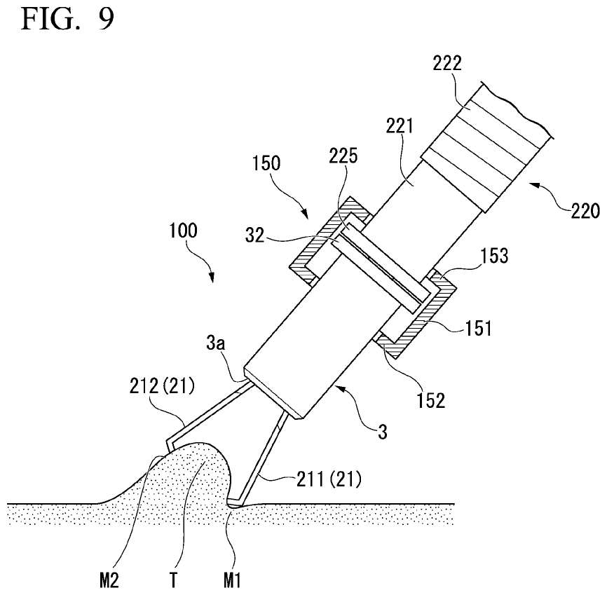

FIG. 9 is a view showing a locking step by the clip unit 100.

The surgeon retracts the manipulation wire 230 by retracting the slider 242 along the manipulation main body 241. When the clip 2 is pulled into a predetermined position on the retaining pipe 3, the engaging portions 25, which is a locking mechanism, engage with the inner circumferential surface of the retaining pipe 3, locking the pair of arms 21 into a closed state. Once the pair of arms 21 are locked in the closed state, the pair of arms 21 cannot return to the open state.

### Separation Step

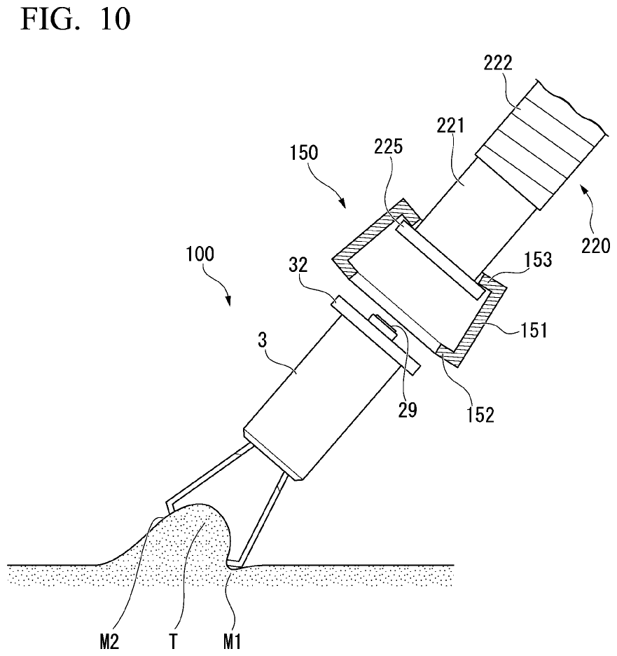

FIG. 10 is a view showing a separating step by the clip unit 100.

The surgeon further pulls the clip 2. A breaking force by tension is applied to the separating portion 29 of the clip 2, causing the separating portion 29 to break. As a result, the clip 2 and the curved member 4 are separated. Further, the deformation of the coupler 150 causes the retaining pipe 3 and the sheath 220 to separate. The surgeon retracts the sheath 220 and leaves the clip 2 and the retaining pipe 3 in the body, ligating the first mucous membrane M1 and the second mucous membrane M2.

According to the clip device 300 of the embodiment, the tissue T can be suitably seized in the lumen. Even when treating the tissue T by the tangential approach, the clip unit 100 can be swung relative to the sheath 220, and the pair of arms 21 can be disposed facing the front surface of the tissue T, which is the treatment region. The clip unit 100 swings relative to the sheath 220, so that the clip unit 100 can be easily disposed to face the front surface of the tissue T even in a narrow lumen.

Hereinafter, the first embodiment of the present invention has been described in detail with reference to the drawings, but the specific configuration is not limited to this embodiment, and design changes that do not deviate from the spirit of the present invention are also included.

### (Variant 1-1)

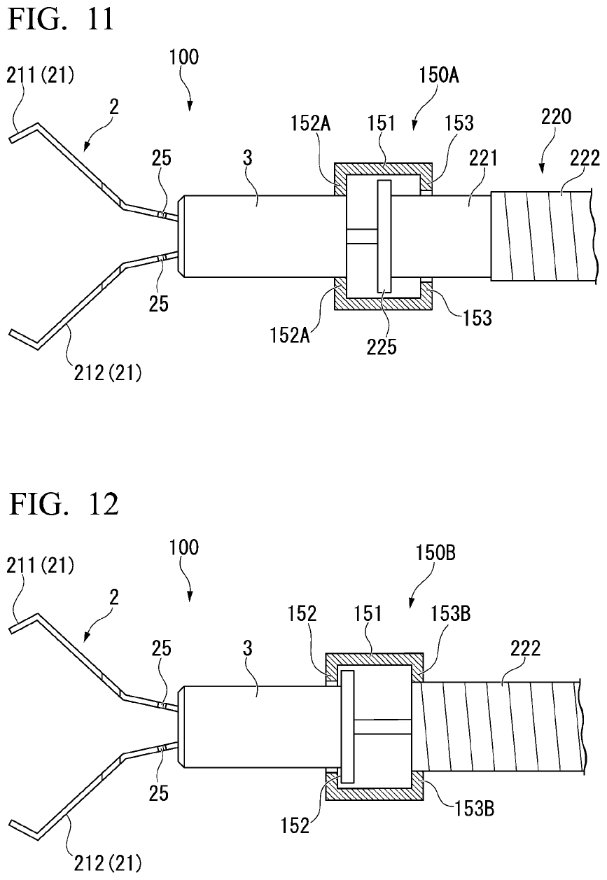

FIG. 11 is a view showing a coupler 150A, which is a variant of the coupler 150.

The coupler 150A has a cylindrical portion 151, a distal end engaging portion 152A, and a proximal end engaging portion 153. The distal end engaging portion 152A is undetachably attached to the retaining pipe 3. The retaining pipe 3 and the coupler 150A may be formed integrally with each other.

### (Variant 1-2)

FIG. 12 is a view showing a coupler 150B, which is a variant of the coupler 150.

The coupler 150B has a cylindrical portion 151, a distal end engaging portion 152, and a proximal end engaging portion 153B. The proximal end engaging portion 153B is undetachably attached to the distal end-side coil 222 of the sheath 220. The distal end-side coil 222 of the sheath 220 and the coupler 150B may be formed integrally with each other.

### (Variant 1-3)

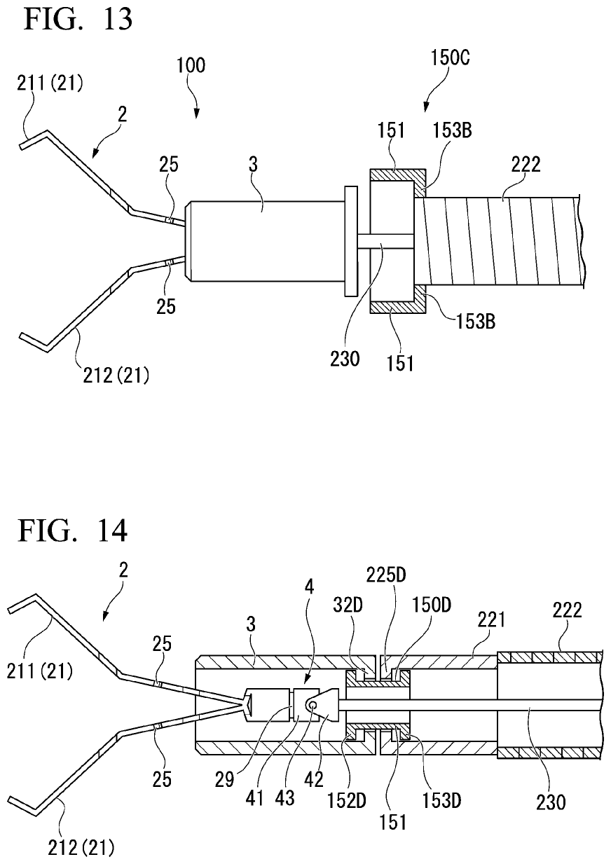

FIG. 13 is a view showing a coupler 150C, which is a variant of the coupler 150.

The coupler 150C has a cylindrical portion 151, and a proximal end engaging portion 153B. The coupler 150C does not have the distal end engaging portion 152. The distal end-side coil 222 of the sheath 220 and the coupler 150C may be formed integrally with each other.

### (Variant 1-4)

FIG. 14 is a view showing a coupler 150D, which is a variant of the coupler 150.

The coupler 150 of the above-mentioned embodiment is disposed outside the retaining pipe 3 and the distal end tip 221 in the radial direction R. Meanwhile, the coupler 150D is disposed inside the retaining pipe 3 and the distal end tip 221 in the radial direction R. The coupler 150D has a cylindrical portion 151, a distal end engaging portion 152D, and a proximal end engaging portion 153D.

The distal end engaging portion 152D is provided at the distal end of the cylindrical portion 151 and is a convex portion that protrudes outward in the radial direction R perpendicular to the longitudinal direction A. The distal end engaging portion 152D engages with the engaging portion 32D, which protrudes inward in the radial direction R at the proximal end of the retaining pipe 3.

The proximal end engaging portion 153D is provided at the proximal end of the cylindrical portion 151 and is a convex portion that protrudes outward in the radial direction R perpendicular to the longitudinal direction A. The proximal end engaging portion 153D engages with the engaging portion 225D, which protrudes inward in the radial direction R at the distal end of the sheath 220.

### Second Embodiment

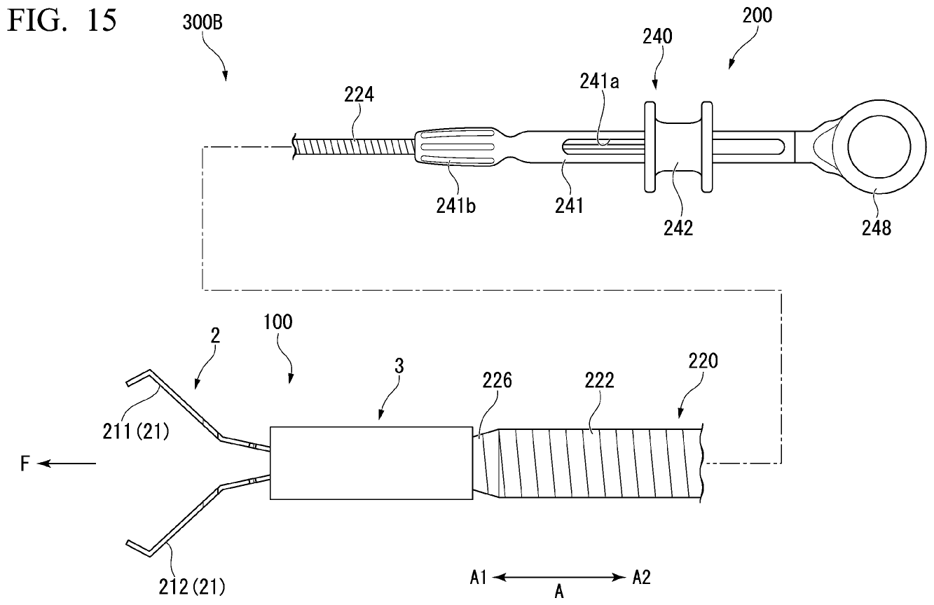

A second embodiment of the present invention will be described with reference to FIG. 15 to FIG. 21. In the following description, the same components as those already described will be given the same reference numerals and redundant explanations will be omitted.

FIG. 15 is a view showing a clip device 300B according to the embodiment.

The clip device (a clip system, a treatment tool for endoscope) 300B includes a clip unit 100, and an applicator 200B. The clip unit 100 is loaded into the applicator 200B, which can be inserted into the channel of the endoscope, and introduced into the treatment location.

The applicator (clip unit introducing device) 200B includes a sheath 220B, a manipulation wire 230, and a manipulation part 240. The applicator 200B, like the applicator 200 of the first embodiment, is inserted into, for example, a treatment tool insertion channel of an endoscope and used in combination with the endoscope.

The sheath 220B differs from the sheath 220 of the first embodiment in the distal end portion. The sheath 220B has a distal end tapered portion 226, a distal end-side coil 222, and a hand-side coil 224, and is formed into an elongated tubular shape as a whole. The distal end tapered portion 226 is located on the side of the distal end portion of the sheath 220.

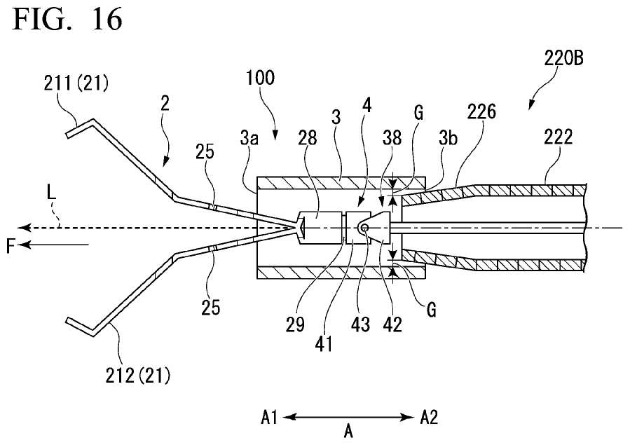

FIG. 16 is a cross-sectional view of the distal end portion of the clip device 300B.

The distal end tapered portion 226 is inclined with respect to the longitudinal axial direction L of the sheath 220 and is formed into a tapered form that decreases in diameter toward the distal end side A1. The retaining pipe 3 and the distal end tapered portion 226 are fitted together. Specifically, the distal end portion of the distal end tapered portion 226 is inserted into the internal space 38 of the retaining pipe 3 from a proximal end opening 3b of the retaining pipe 3.

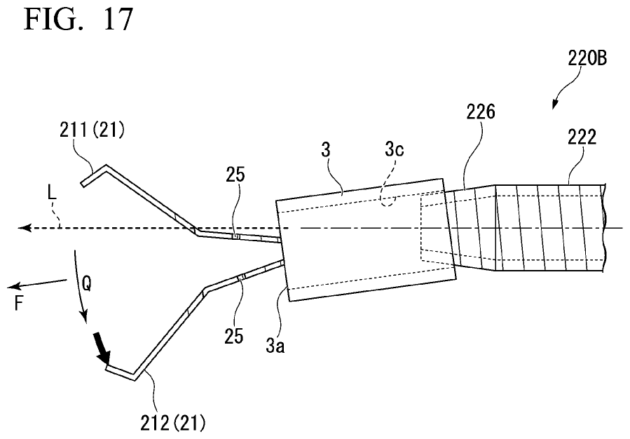

FIG. 17 is a view showing transition between forms of the clip unit 100.

The clip unit 100 can transition between the linear form (first form) shown in FIG. 16 and the swing form (second form) shown in FIG. 17.

When a force greater than or equal to a predetermined value is applied to at least one of the first arm 211 and the second arm 212, the clip unit 100 swings in the swing direction Q and transitions to the swing form (second form). Specifically, as the pair of arms 21 swing in the swing direction Q, the retaining pipe 3 in contact with the pair of arms 21 moves relative to the sheath 220. When the clip unit 100 is at least in the linear form (first form), the gap (rattle) G in the radial direction R is formed between an inner circumferential surface 3c of the retaining pipe 3 and the distal end tapered portion 226 of the sheath 220. For this reason, the retaining pipe 3 can be inclined relative to a direction that intersects with the longitudinal axis direction L of the sheath 220.

The clip unit 100 can transition from the swing form (second form) to the linear form (first form).

### Operation and Action of Clip Device 300B

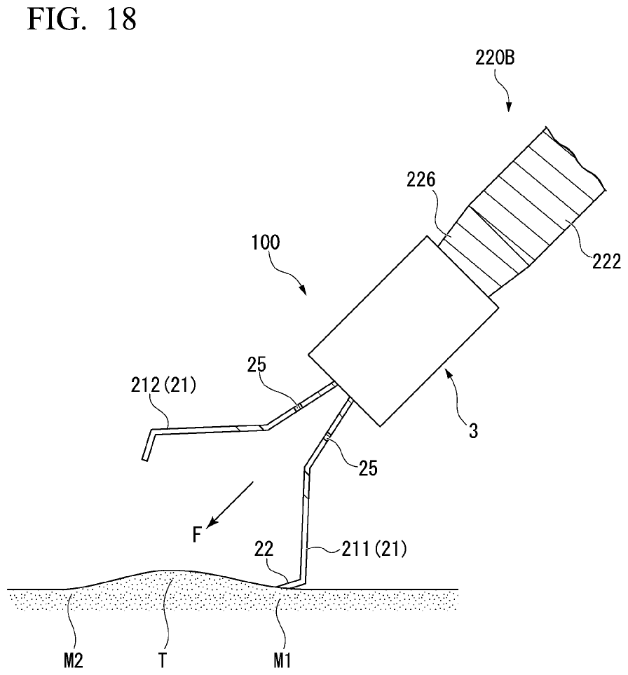

Next, operations and actions of the clip device 300B will be described with reference to FIG. 18 to FIG. 21.

### Approaching Step

The surgeon introduces the clip unit 100 loaded into the applicator 200B into the body via the channel of the endoscope. The surgeon moves the endoscope or the sheath 220B to bring the clip 2 of the clip unit 100 closer to the tissue T, which is the treatment region.

### Tangential Approach

FIG. 18 is a view showing the clip unit 100 configured to treat the tissue T by the tangential approach. The surgeon approaches the tissue T, which is the treatment region, with the clip unit 100 in the linear form by the tangential approach. The surgeon hooks the claw 22 of the first arm 211 onto the mucous membrane on the endoscope side (front side) of the first mucous membrane M1 and the second mucous membrane M2 (the first mucous membrane M1 in FIG. 18), which are located on either side sandwiching the center of the tissue T.

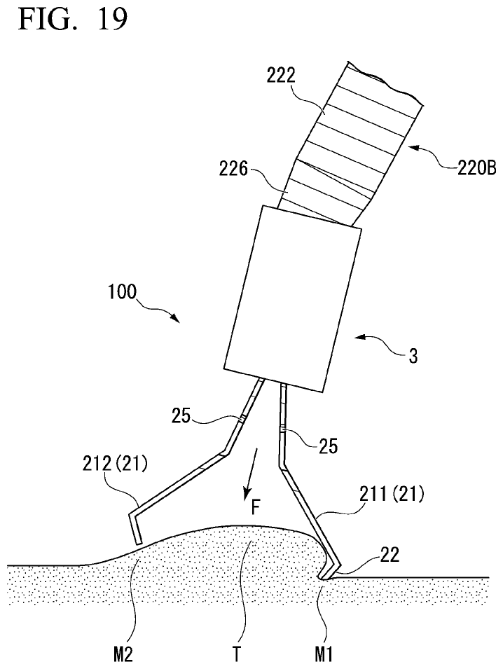

FIG. 19 is a view showing the clip unit 100 with the pair of arms 21 swung. The surgeon brings the clip unit 100, with the claw 22 hooked onto the first mucous membrane M1, closer to the tissue T, which is the treatment region. Specifically, the surgeon advances the applicator 200B against the channel of the endoscope. Alternatively, the surgeon may further press the clip unit 100 against the tissue T by advancing the endoscope. As a result, as shown in FIG. 19, the pair of arms 21 swings around the rotation axis RO, and the clip unit 100 takes on the swing form. When the second arm 212 comes into contact with the second mucous membrane M2, the pair of arms 21 are disposed so that the forward direction F faces the front surface of the tissue T, which is the treatment region. The first mucous membrane M1 and the second mucous membrane M2 are grasped by the pair of arms 21.

### Pulling Step

The user retracts the manipulation wire 230 by retracting the slider 242 along the manipulation main body 241. The curved member 4 connected to the manipulation wire 230 pulls the clip 2. As the clip 2 is pulled to the proximal end side A2, the pair of arms 21 are pulled into the retaining pipe 3, gradually closing the pair of arms 21 that engage with a distal end opening 3a. In this state, when the pulling force of the manipulation wire 230 is released, the self-expanding power of the pair of arms 21 causes the clip 2 to move to the distal end side A1 and return to the open state. The user can return the pair of arms 21 to the open state and grasp the tissue T again (reopening).

### Locking Step

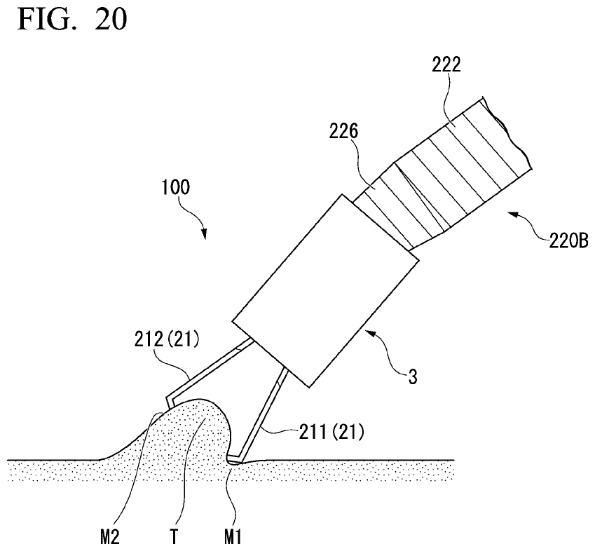

FIG. 20 is a view showing a locking step by the clip unit 100.

The surgeon retracts the manipulation wire 230 by retracting the slider 242 along the manipulation main body 241. When the clip 2 is pulled into a predetermined position on the retaining pipe 3, the engaging portions 25, which is the locking mechanism, engage with the inner circumferential surface of the retaining pipe 3, locking the pair of arms 21 into the closed state. Once the pair of arms 21 are locked in the closed state, the pair of arms 21 cannot return to the open state.

### Separating Step

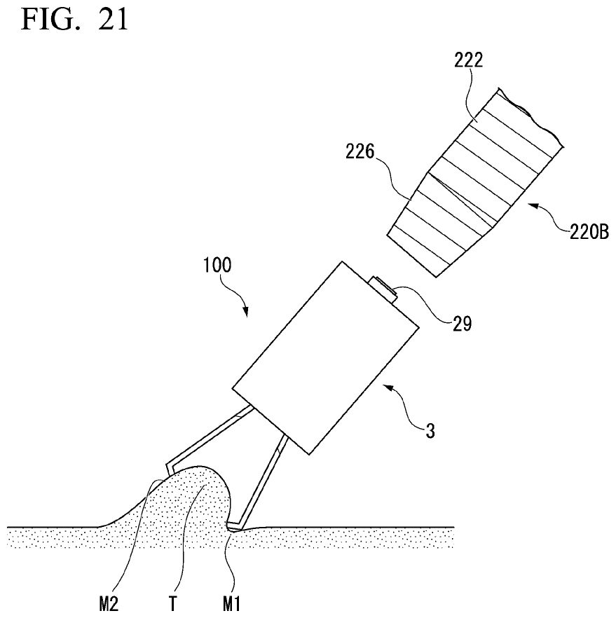

FIG. 21 is a view showing a separating step by the clip unit 100.

The surgeon further pulls the clip 2. A breaking force by tension is applied to the separating portion 29 of the clip 2, causing the separating portion 29 to break. As a result, the clip 2 and the curved member 4 are separated. The surgeon retracts the sheath 220, leaving the clip 2 and the retaining pipe 3 inside the body, which have ligated the first mucous membrane M1 and the second mucous membrane M2.

According to the clip device 300B of the embodiment, the tissue T can be suitably seized in the lumen. Even when treating the tissue T by the tangential approach, the clip unit 100 can be swung relative to the sheath 220B, and the pair of arms 21 can be disposed facing the front surface of the tissue T, which is the treatment region. The clip unit 100 swings relative to the sheath 220B, so that the clip unit 100 can be easily disposed facing the front surface of the tissue T even in a narrow lumen.

Hereinabove, the second embodiment of the present invention has been described in detail with reference to the drawings, but the specific configuration is not limited to this embodiment, and design changes that do not deviate from the spirit of the present invention are also included. In addition, the components shown in the embodiment and variants can be configured by combining them as appropriate.

### (Variant 2-1)

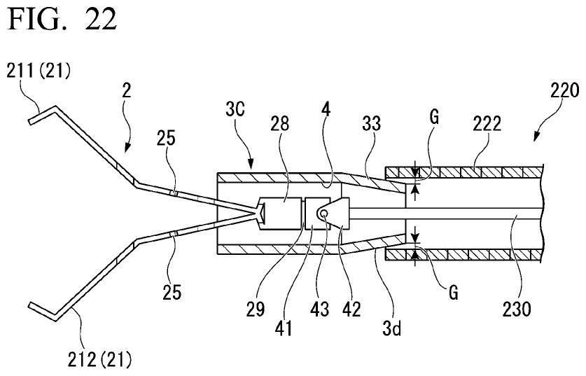

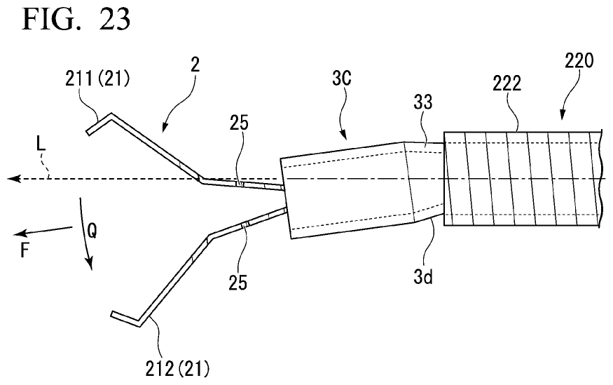

FIG. 22 and FIG. 23 are views showing the retaining pipe 3C, which is a variant of the retaining pipe 3.

The retaining pipe 3C has a proximal end tapered portion 33 on the proximal end side. The proximal end tapered portion 33 is formed in a tapered form that narrows toward the proximal end side A2. A proximal end portion of the proximal end tapered portion 33 is inserted into the sheath 220. When the clip unit 100 is at least in the linear form (first form), the gap (rattle) G in the radial direction R is formed between an outer circumferential surface 3d of the retaining pipe 3C and the inner circumferential surface of the sheath 220. For this reason, the retaining pipe 3C can be inclined relative to a direction that intersects with the longitudinal axis direction L of the sheath 220.

### (Variant 2-2)

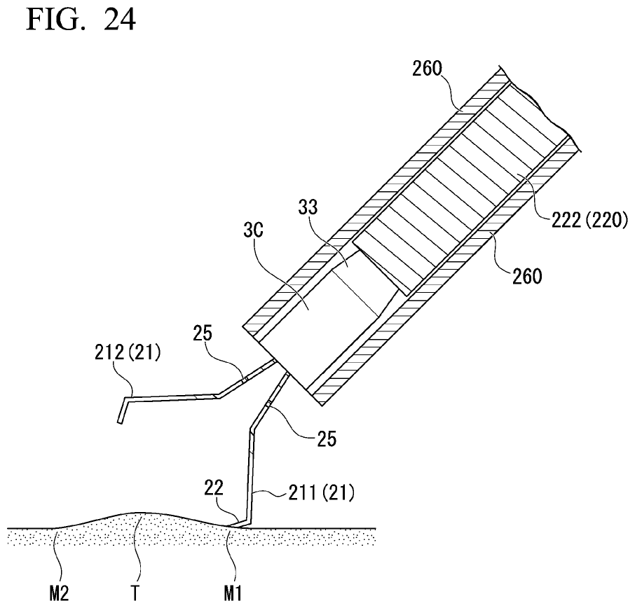

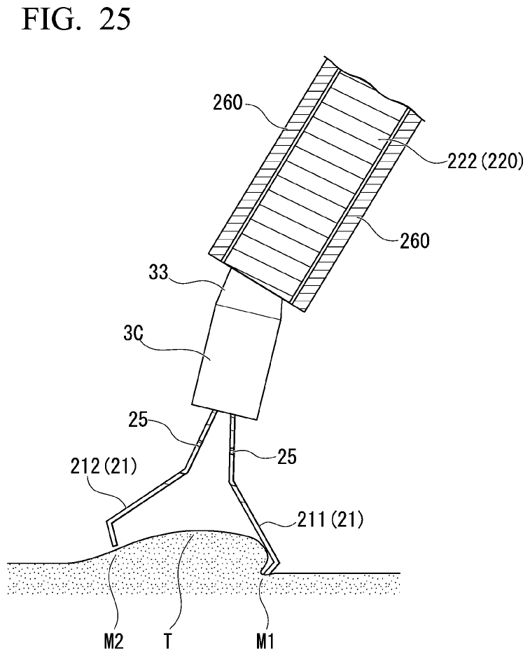

FIG. 24 to FIG. 27 are views showing a procedure using an outer sheath 260. The applicator 200 may further include the outer sheath 260. As shown in FIG. 24, by disposing the outer sheath 260 on the outside of the sheath 220 and the retaining pipe 3C, the outer sheath 260 can maintain the clip unit 100 in the linear form (first form). As shown in FIG. 25, by protruding the retaining pipe 3C from the outer sheath 260 to the distal end side A1, the clip unit 100 can transition to the swing form (second form). As shown in FIG. 26 and FIG. 27, with the retaining pipe 3C protruded from the outer sheath 260 to the distal end side A1, the locking step and the separating step are performed.

### Third Embodiment

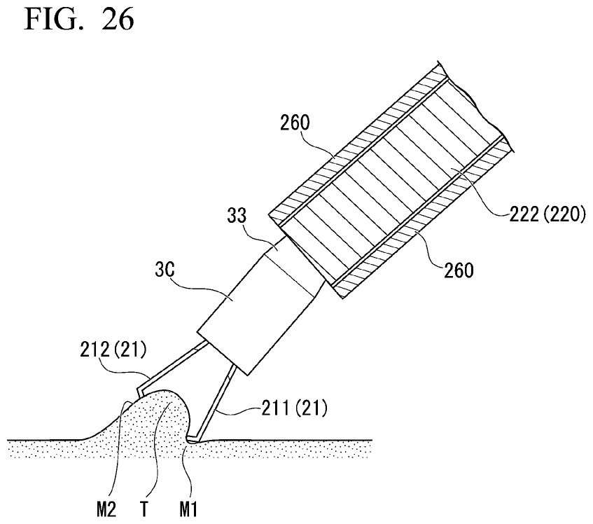

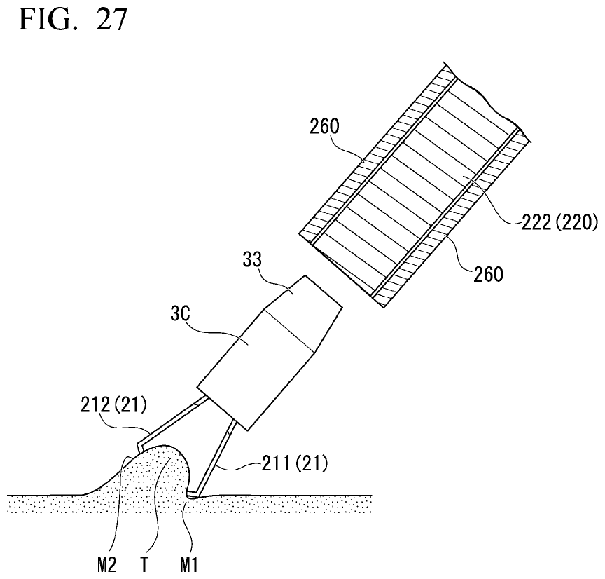

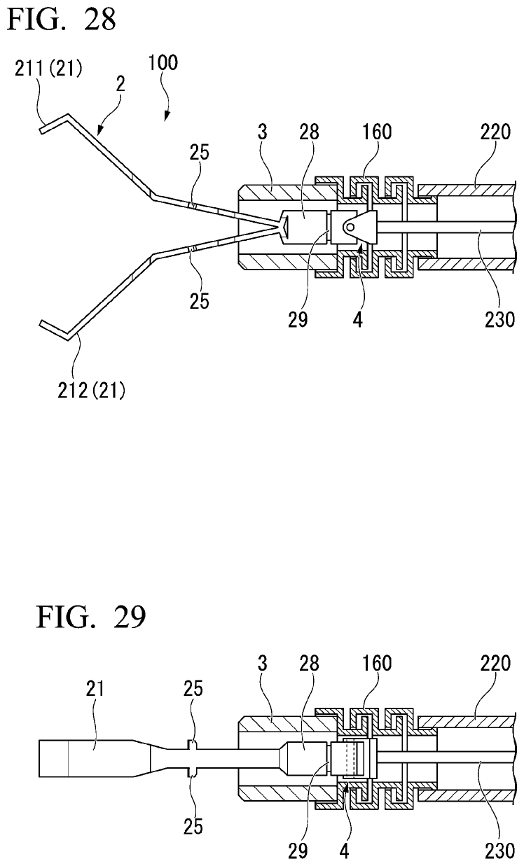

A third embodiment of the present invention will be described with reference to FIG. 28 to FIG. 31. In the following description, the same components as those already described will be given the same reference numerals and redundant explanations will be omitted.

FIG. 28 and FIG. 29 are views showing a clip device 300C according to the embodiment.

The clip device (a clip system, a treatment tool for endoscope) 300C includes a clip unit 100, a flexible member 160, and an applicator 200.

The flexible member 160 detachably connects the retaining pipe 3 and the sheath 220. The flexible member 160 is formed in a tubular shape and has flexibility. The flexible member 160 is, for example, bellows, a tube, or a laser spiral cut pipe. Since the flexible member 160 has flexibility, the retaining pipe 3 can be inclined relative to a direction intersecting with the longitudinal axis direction L of the sheath 220.

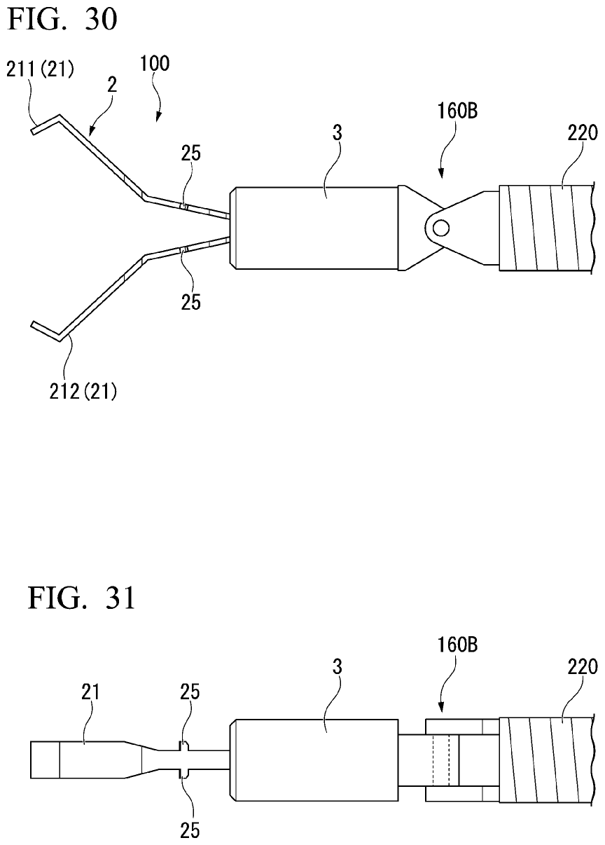

FIG. 30 and FIG. 31 are views showing a flexible member 160B, which is another aspect of the flexible member 160. The flexible member 160B detachably connects the retaining pipe 3 and the sheath 220. The flexible member 160 is, for example, a hinge structure. Since the flexible member 160B has flexibility, the retaining pipe 3 can be inclined relative to a direction intersecting with the longitudinal axis direction L of the sheath 220.

According to the clip device 300C of the embodiment, the tissue T can be suitably seized in the lumen. Even when treating the tissue T by the tangential approach, the clip unit 100 can be swung relative to the sheath 220, and the pair of arms 21 can be disposed facing the front surface of the tissue T, which is the treatment region. The clip unit 100 swings relative to the sheath 220, so that the clip unit 100 can be easily disposed facing the front surface of the tissue T even in a narrow lumen.

Hereinabove, the third embodiment of the present invention has been described in detail with reference to the drawings, but the specific configuration is not limited to this embodiment, and design changes within the scope that do not deviate from the spirit of the present invention are also included. In addition, the components shown in the embodiment and variants can be configured by combining them as appropriate.

### (Variant 3-1)

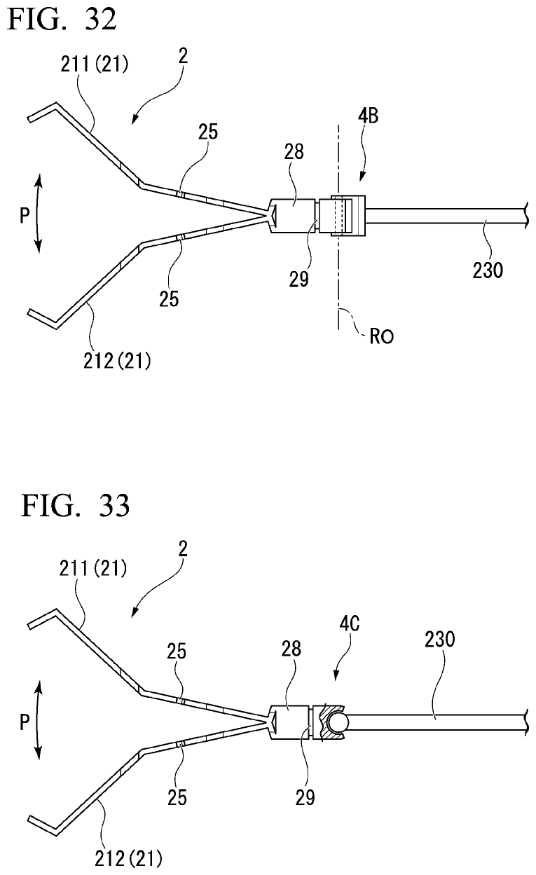

FIG. 32 is a view showing a curved member 4B, which is a variant of the curved member 4.

The curved member 4B supports the pair of arms 21 so that they can swing (pivot) around the rotation axis RO, which is approximately perpendicular to the longitudinal direction A. The swing direction (rotation direction) Q of the pair of arms 21, with the rotation axis RO as the center of rotation, is approximately perpendicular to the opening/closing direction P of the pair of arms 21. In this case, the clip unit can transition between the linear form (first form) in which the forward direction F of the pair of arms 21 faces the same direction as the longitudinal axis direction L, and the swing form (second form) in which the clip unit swings in a direction approximately perpendicular to the opening/closing direction P, so that the forward direction F of the pair of arms 21 faces in a direction that intersects with the longitudinal axis direction L.

FIG. 33 is a view showing a curved member 4C, which is a variant of the curved member 4.

The curved member 4C is a ball joint that supports the pair of arms 21 so that they can swing (pivot) around a ball 43C. The swing direction (rotation direction) Q of the pair of arms 21 with the ball 43C as the center of rotation is omni-directional. In this case, the clip unit can transition between the linear form (first form) in which the forward direction F of the pair of arms 21 faces the same direction as the longitudinal axis direction L, and the swing form (second form) in which it swings in all directions and faces in a direction that intersects with the forward direction F of the pair of arms 21.

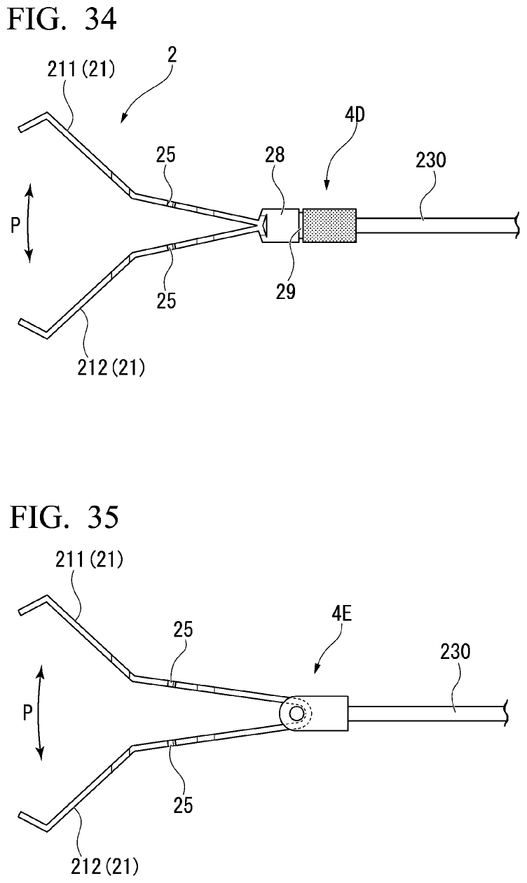

FIG. 34 is a view showing a curved member 4D, which is a variant of the curved member 4.

The curved member 4D is an elastic member such as rubber. The swing direction (rotation direction) Q of the pair of arms 21 with the curved member 4D as the center of rotation is omni-directional. In this case, the clip unit can transition between the linear form (first form) in which the forward direction F of the pair of arms 21 faces the same direction as the longitudinal axis direction L, and the swing form (second form) in which it swings in all directions and faces in a direction that intersects with the forward direction F of the pair of arms 21.

FIG. 35 is a view showing a curved member 4E, which is a variant of the curved member 4.

The curved member 4E is a hook provided at the distal end of the manipulation wire 230. The swing direction (rotation direction) Q of the clip 2 substantially coincides with the opening/closing direction P of the pair of arms 21 like the first embodiment. In this case, the clip unit can transition between the linear form (first form) in which the forward direction F of the pair of arms 21 faces the same direction as the longitudinal axis direction L, and the swing form (second form) in which the forward direction F of the pair of arms 21 swings in approximately the same direction as the opening/closing direction P and faces in a direction that intersects with the longitudinal axis direction L.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A clip device comprising:
a clip unit having arms configured to be opened and closed;
a wire connected to the clip unit; and
a sheath through which the wire is inserted and extending in a longitudinal axis direction,
wherein the clip unit is detachably connected to a distal end portion of the sheath, and
the clip unit is able to transition between:
a first form in which the clip unit is configured to be opened and closed around a central axis of the sheath, and
a second form in which the clip unit is configured to be opened and closed around an axis line intersecting the central axis.

2. The clip device according to claim 1, comprising a connecting member configured to detachably connect the clip unit and the distal end portion of the sheath,
wherein, when the clip unit is in the first form, a gap is formed in the longitudinal axial direction between the clip unit and the distal end portion of the sheath connected by the connecting member.

3. The clip device according to claim 2, wherein the gap has a first gap and a second gap,
the first gap and the second gap are disposed on either side sandwiching the wire in a radial direction of the wire,
when the clip unit is in the first form, a first distance of the first gap and a second distance of the second gap are the same, and
when the clip unit is in the second form, the first distance and the second distance are different from each other.

4. The clip device according to claim 3, wherein, when the clip unit is transitioned from the first form to the second form, one of the first distance and second distance increases.

5. The clip device according to claim 4, wherein, when the clip unit is transitioned from the first form to the second form, the other of the first distance and the second distance decreases.

6. The clip device according to claim 2, wherein the connecting member is undetachably attached to the clip unit.

7. The clip device according to claim 2, wherein the connecting member is undetachably attached to the distal end portion of the sheath.

8. The clip device according to claim 2, wherein the clip unit has a tubular member capable of storing at least a portion of the arms, and
the connecting member detachably connects the tubular member to the distal end portion of the sheath.

9. The clip device according to claim 1, wherein the clip unit has a tubular member capable of storing at least a portion of the arms,
the distal end portion of the sheath and the tubular member are fitted together, and
when the clip unit is in the first form, a radial gap is formed between the tubular member and the distal end portion of the sheath.

10. The clip device according to claim 9, wherein at least a portion of a tapered portion formed on the distal end portion of the sheath is inserted into the tubular member.

11. The clip device according to claim 9, wherein at least a portion of a tapered portion formed on a proximal end portion of the tubular member is inserted into the distal end portion of the sheath.

12. The clip device according to claim 1, comprising a flexible member configured to detachably connect the clip unit and the distal end portion of the sheath.

13. The clip device according to claim 1, wherein the clip unit has a tubular member capable of storing at least a portion of the arms, and
further has an outer sheath disposed outside the tubular member and the distal end portion of the sheath.

14. The clip device according to claim 1, wherein a swing direction of the clip unit transitioning between the first form and the second form is the same as an opening/closing direction of the arms.

15. The clip device according to claim 1, wherein a swing direction of the clip unit transitioning between the first form and the second form is perpendicular to an opening/closing direction of the arms.

16. The clip device according to claim 1, wherein a swing direction of the clip unit transitioning between the first form and the second form is omni-direction.
